(19) United States
(12) Patent Application Publication
(10) Pub. No.: US 2010/0248325 A1
(43) Pub. Date: Sep. 30, 2010

(54) SELF-CROSSLINKING POLYSILOXANES IN COATINGS OF ENZYME IMMOBILIZATES

(75) Inventors: Marrit Eckstein, Essen (DE); Michael Ferenz, Essen (DE); Oliver Thum, Ratingen (DE)

Correspondence Address: SCULLY SCOTT MURPHY & PRESSER, PC, 400 GARDEN CITY PLAZA, SUITE 300, GARDEN CITY, NY, 11530, US

(73) Assignee: EVONIK GOLDSCHMIDT GMBH, Essen (DE)

(21) Appl. No.: 12/750,813

(22) Filed: Mar. 31, 2010

(30) Foreign Application Priority Data
Mar. 31, 2009 (DE) 10 2009 015 211.3

(51) Int. Cl. C12N 11/08; C12N 11/00

(52) U.S. Cl. 435/180; 435/174

(57) ABSTRACT

Enzyme preparations obtainable by providing an enzyme immobilizate with a silicone coating, the coating being obtained by hydrosilylating a self-crosslinking polysiloxane, a process for production thereof and the use thereof are provided.

### FIELD OF THE INVENTION

The invention relates to novel enzyme preparations, to the use of such enzyme preparations as biocatalysts and to a process for production of enzyme preparations.

### BACKGROUND OF THE INVENTION

Microorganisms and isolated enzymes find wide use as a catalyst in the chemical industry or in food production. An overview is offered, for example, by: A. Liese, K. Seelbach, C. Wandrey, Industrial Biotransformations, Wiley-VCH: 2000, Weinheim, Germany.

In order to ensure economic use of such biocatalysts, some conditions have to be satisfied: the biocatalyst has to be active for a sufficiently long time under the reaction conditions, the biocatalyst should be readily removable after the end of the reaction and the biocatalyst should be reusable as often as possible. Ideally, these conditions should be satisfied for a very wide range of reaction conditions (for example temperature range, type of solvents used, pressures, etc.), in order to provide as universal as possible a catalyst.

In order to satisfy these conditions, it is typically necessary to immobilize the enzymes or microorganisms comprising the enzymes used.

Frequently, the enzymes or microorganisms comprising enzymes are immobilized noncovalently on supports; the supports used are frequently ion exchange resins or polymer particles which possess suitable particle size distributions. Examples for this purpose are the commercial products Novozym 435, Lipozym RM IM or Lipozym TL IM from Novozymes A/S, Bagsvaerd, Denmark or Amano PS, from Amano, Japan. These examples are immobilized lipases which find wide use, since such immobilizates also exhibit industrially utilizable activities in nonaqueous systems, i.e., those which comprise only organic solvents, if any, as described, for example, in J. Chem. Soc., Chem. Comm. 1989, 934-935. Patent application EP 2 011 865 describes the various disadvantages of the presently available immobilization technologies, in particular with regard to activity and stability.

At the same time, the prior art EP application teaches a novel class of enzyme preparations and a process for preparing them which overcomes a large portion of these disadvantages. Essentially, it is stated in the aforementioned patent application that stable and highly active enzyme preparations can be obtained by first immobilizing enzymes or microorganisms comprising enzymes on a suitable support and then providing this immobilizate with a silicone polymer which is obtainable by a hydrosilylation reaction.

In the aforementioned EP application, in a two-component system, preferably SiH-functionalized polysiloxanes are reacted in the presence of a catalyst, preferably a transition metal catalyst, with organomodified polysiloxanes which possess at least one terminal carbon-carbon double bond, preferably at least two double bonds.

In such a two-component system, in addition to the structure of the siloxanes used, the stoichiometry of the siloxanes used also decides the properties of the siloxane coating, which has a strong influence on the quality of the enzyme preparations thus obtained. Disadvantages of this prior art method arise from impaired manageability, for example as a result of possible weighing errors which cause an incorrect stoichiometry and hence unforeseeable properties of the silicone coating and of the activity and stability of the immobilizates.

Furthermore, the stoichiometry of the siloxanes used decides the quality of the immobilizates when the two-component mixture, as described in patent application EP 2 011 865 A1, is sprayed into a fluidized bed reactor or onto a pelletizing pan, which significantly impairs the automated silicone coating of immobilizates. To ensure the desired quality, especially the homogeneous distribution of the siloxane components in terms of time and space, specifically in the case of scale-up, for example pilot or production scale, a particularly high level of apparatus complexity is needed, which can be reflected in high capital, operating and maintenance costs.

### SUMMARY OF THE INVENTION

The present invention provides enzyme immobilizates which have comparable good properties with simplified and reproducible production requirements compared to the enzyme immobilizates known from the prior art.

More particularly, the present invention provides an enzyme preparation obtainable by providing an enzyme immobilizate with a silicone coating, the coating being obtained by hydrosilylating a self-crosslinking polysiloxane.

The present invention also provides a process for producing the inventive enzyme preparation as well as for the use thereof.

### DESCRIPTION OF THE INVENTION

The present invention provides a self-crosslinking siloxane to coat enzyme immobilizates.

More particularly, and as stated above, the present invention provides an enzyme preparation obtainable by providing an enzyme immobilizate with a silicone coating, the coating being obtained by hydrosilylating a self-crosslinking polysiloxane.

The term “a self-crosslinking polysiloxane” is understood in the context of the present invention to mean a polysiloxane which, in the course of a hydrosilylation reaction, can form intermolecular covalent bonds with itself, for example in the simultaneous presence of SiH and terminally unsaturated alkyl groups.

The term “enzyme immobilizate” in the context of the present invention is understood to mean an inert support comprising at least one enzyme or microorganisms comprising enzymes, the freedom of movement of which is restricted, i.e., it or they are “immobilized”. The enzyme can be immobilized by various interactions with the material of the inert support, for example, covalent bonding, van der Waals interactions, physical inclusion, etc., but it is also conceivable that the enzyme is immobilized indirectly with the material of the inert support by means of at least one further bridging element, for example, using coupling reagents, such as, for example, glutaraldehyde.

One advantage of the present invention is that only one raw material is required for coating. A further advantage is the clear simplification of the management and of the apparatus complexity in the silicone coating process.

Another advantage of the present invention is that the enzyme immobilizates have homogeneous and reproducible silicone coatings.

The inventive enzyme preparations and a process for production thereof are described below by way of example, without any intention that the invention be restricted to these illustrative embodiments. When ranges, general formulae or compound classes are specified below, these shall not only encompass the corresponding ranges or groups of compounds which are mentioned explicitly but also all sub-ranges and sub-groups of compounds which can be obtained by selecting individual values (ranges) or compounds. When documents are cited within the present description, their contents shall be incorporated completely in the disclosure-content of the present invention. When compounds, for example organically modified polysiloxanes, which may have different units more than once are described in the context of the present invention, they may occur in these compounds in random distribution (statistical oligomer) or ordered (block oligomer). Figures for the number of units in such compounds should be interpreted as the mean value, averaged over all appropriate compounds.

Preferred inventive enzyme preparations use, as the self-crosslinking polysiloxane, compounds of the general formula (I)

MₐM^{H}_{b}M^{V}_{c}M^{R}_{d}DₑD^{H}_{f}D^{V}_{g}D^{R}ₕTᵢT^{H}ⱼT^{V}ₖT^{R}ₗQₘ (I)

where
M=[R¹₃SiO_{1/2}],
M^{H}=[HR¹₂SiO_{1/2}],
M^{V}=[R²R¹₂SiO_{1/2}],
M^{R}=[R³R¹₂SiO_{1/2}],
D=[R¹₂SiO_{2/2}],
D^{H}=[HR¹SiO_{2/2}],
D^{V}=[R²R¹SiO_{2/2}],
D^{R}=[R³R¹SiO_{2/2}],
T=[R¹SiO_{3/2}],
T^{H}=[HSiO_{3/2}],
T^{V}=[R²SiO_{3/2}],
T^{R}=[R³SiO_{3/2}], and
Q=[SiO_{4/2}]
   where
R¹=independently identically or differently, selected from the group comprising: saturated or unsaturated, unbranched or branched alkyl groups having 1 to 30 carbon atoms, alkaryl radicals having 7 to 30 carbon atoms, aryl radicals having 6 to 30 carbon atoms, preferably alkyl groups having 1 to 4 carbon atoms or phenyl, especially methyl,
R²=identical or different radicals of the general formula (II)

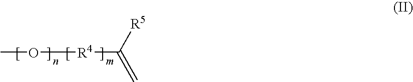

where
R⁴=identical or different, linear or branched alkanediyl radicals, optionally interrupted by one or more ether functions, preferably —(CH₂)₄—, —(CH₂)—, —(CH₂)₃—O—CH₂—, —(CH₂)₂—[C₂H₄O]ₒ[C₃H₆O]ₚ—CH₂— and —(CH(CH3)CH₂)—[(C₂H₄O]ₒ[C₃H₆O]ₚ—CH₂—,
   where
o, p=1 to 500, preferably 1 to 150,
R⁵=R¹ or H, preferably H,
where
n, m=0 or 1, preferably 0,
R³=identical or different polyether radicals, preferably identical or different polyether radicals of the general formula (III)

—CH₂CH₂CH₂—[CH₂CH₂O]_{q}[CH₂CH(CH₃)O]ᵣ[CH₂CH(C₂H₅)O]ₛ[CH₂CH(Ph)O]ₜ—R⁶ (III)

where
R⁶=identical or different radicals from the group of
H, R¹ and —C(O)—R¹
where q, r, s, t=0 to 50, preferably q, r, =1 to 50 and s, t=0, especially q, r=3 to 50 and s, t=0,
   where
a=0-30, preferably 0-12, especially 0-2,
b=0-30, preferably 0-12, especially 0-2,
c=0-30, preferably 0-12, especially 0-2,
d=0-30, preferably 0-12, especially 0-2,
e=0-800, preferably 0-600, especially 10-400,
f=0-30, preferably 0-20, especially 0-10,
g=0-30, preferably 0-20, especially 0-10,
h=0-40, preferably 0-10, especially 0,
i=0-10, preferably 0-5, especially 0,
j=0-10, preferably 0-5, especially 0,
k=0-10, preferably 0-5, especially 0,
l=0-10 preferably 0-5, especially 0, and
m=0-10, preferably 0-5, especially 0,
with the proviso that
b+f+j>1, preferably >1.5, especially >1.9,
and
c+g+h>1, preferably >1.5, especially >1.9.

The provisos b+f+j>1 and c+g+h>1 describe the fact that each siloxane molecule statistically possesses both more than one SiH function and more than one hydrosilylatable carbon-carbon double bond. This achieves the effect that the siloxanes used in the context of this invention are self-crosslinking and it is therefore necessary to only use one siloxane component for coating.

In some cases, it may be advisable to mix two or more self-crosslinking polysiloxanes of the general formula (I) with one another or to use them successively, or to apply two or more layers comprising different siloxanes of the general formula (I).

In a particular embodiment, the self-crosslinking polysiloxanes used are compounds of the general formula (I) in which
a, b, d, g, h, i, j, k, l, m=0,
c=2,
e=10 to 800, preferably 30 to 600,
f=1 to 20, preferably 2 to 12,
R¹=alkyl, preferably methyl, and
R²=a terminally unsaturated alkyl or alkoxy radical, preferably vinyl.

In a further embodiment, the self-crosslinking polysiloxanes used are compounds of the general formula (I) in which
b, c, d, h, i, j, k, l, m=0,
a=2,
e=10 to 800, preferably 30 to 600,
f=1 to 20, preferably 2 to 12,
g=1 to 20, preferably 2 to 12,
R¹=alkyl, preferably methyl, and
R²=a terminally unsaturated alkyl or alkoxy radical, preferably vinyl.

In a further embodiment, the self-crosslinking polysiloxanes used are compounds of the general formula (I) in which
a, b, d, g, h, j, k, l, m=0,
c=3 to 20, preferably 3 to 12,
e=10 to 800, preferably 30 to 600,
f=1 to 20, preferably 2 to 12, and
i=1 to 18, preferably 1 to 10.

The inventive enzyme preparations can be produced analogously to the process described in patent application EP 2 011 865 A1, optionally in the presence of a catalyst and optionally in the presence of suitable solvents, the self-crosslinking polysiloxanes being used as one-component system for the coating of enzyme immobilizates by hydrosilylation reaction.

To produce the enzyme immobilizates, it is possible to use whole cells, resting cells, purified enzymes or cell extracts which comprise the corresponding enzymes, or mixtures thereof. Preference is given to using hydrolytic enzymes, for example lipases, esterases or proteases, for example lipases from Candida rugosa, Candida antarctica, Pseudomonas sp., Thermomyees lanuginosus, porcine pancreas, Mucor miehei, Alcaligenes sp., cholesterol esterase from Candida rugosa, esterase from the porcine liver, more preferably lipases. Accordingly, the enzyme immobilizates preferably comprise enzymes from the class of the hydrolases, preferably lipases.

The inert supports used may be inert organic or inorganic supports. The inert supports used, or present in the enzyme immobilizate, are preferably those particulate supports which have a particle size distribution in which at least 90% of the particles have a particle size of 10 to 5000 μm, preferably of 50 μm to 2000 μM. The organic supports used may especially be those which comprise, or consist of, polyacrylate, polymethacrylate, polyvinylstyrene, styrene-divinylbenzene copolymers, polypropylene, polyethylene, polyethylene terephthalate, PTFE and/or other polymers. The support materials used may, depending on the enzyme to be immobilized, especially be acidic or basic ion exchange resins, for example Duolite A568, Duolite XAD 761, Duolite XAD 1180, Duolite XAD 7HP, Amberlite IR 120, Amberlite IR 400, Amberlite CG 50, Amberlyst 15 (all products from Rohm and Haas) or Lewatit CNP 105 and Lewatit VP OC 1600 (products from Lanxess, Leverkusen, Germany). The inorganic supports used may be oxidic and/or ceramic supports known from the prior art. In particular, the inorganic supports used may, for example, be Celite, zeolites, silica, controlled-pore glass (CPG) or other supports, as described, for example, in L. Cao, “Carrier-bound Immobilized Enzymes: Principles, Application and Design”, Wiley-VCH: 2005, Weinheim, Germany. More preferably, the inert supports present in the enzyme immobilizate or the inert supports used to produce the enzyme immobilizates comprise polyvinylstyrene, polymethacrylate or polyacrylate. In one embodiment, the inert supports preferably consist of the polymers mentioned hereinabove.

The immobilization on the particles can be effected covalently or noncovalently, preferably noncovalently. For noncovalent immobilization, the support can be incubated or impregnated, for example, with an aqueous enzyme solution which may optionally comprise further constituents, for example inorganic salts or detergents. This incubation/impregnation can be carried out, for example, at temperatures between 0° C. and 50° C., preferably between 0° C. and 40° C. Preference is given to effecting the incubation/impregnation over a period of a few minutes to a few hours. The progress of the incubation can be effected by determining the concentration of the enzyme in the solution with the common methods for protein determination. On attainment of the desired degree of immobilization, the support can preferably be washed with water and, if desired, dried. An enzyme immobilizate obtained in this way can subsequently be provided with a silicone coating in accordance with the invention.

According to the invention, it is, however, also possible to use enzyme immobilizates which are commercially available, for example Novozym 435, Lipozym RM IM or Lipozym TL IM from Novozymes A/S, Bagsvaerd, Denmark, or Amano PS from Amano, Japan.

The hydrosilylation can be carried out by established methods in the presence of a catalyst. It is possible, for example, to use catalysts which are typically used for hydrosilylations, for example platinum, rhodium, osmium, ruthenium, palladium, iridium complexes or similar compounds or the corresponding pure elements or their derivatives immobilized on silica, alumina or activated carbon or similar support materials. Preference is given to performing the hydrosilylation in the presence of Pt catalysts such as cisplatin or Karstedt catalyst [tris(divinyltetramethyldisiloxarie)bis-platinum].

The amount of catalyst used is preferably 10⁻⁷ to 10⁻¹ mol per mole of olefin or per mole of terminal carbon-carbon double bond, preferably 1 to 100 ppm. The hydrosilylation is carried out preferably at temperatures of 0° C. to 200° C., preferably of 20° C. to 120° C.

The hydrosilylation can be carried out in the presence or absence of solvents. Generally, solvents are not needed for the performance of the reaction. The reaction can, however, be carried out in suitable solvents, including for example aliphatic or aromatic hydrocarbons, cyclic oligosiloxanes, alcohols or esters. Suitable solvents are especially cyclohexane or toluene.

According to the invention, based on the mass of the support used, preferably 1 to 500% by mass, preferentially 10 to 200% by mass, more preferably 20 to 150% by mass, of siloxane components of the general formula (I) are used.

The inventive enzyme preparations, produced by hydrosilylation, can be obtained by carrying out the hydrosilylation in the presence of the enzyme immobilizates. However, it is also possible to obtain the coatings by subsequently applying a siloxane obtained by hydrosilylation to the enzyme immobilizates. This can be done, for example, by treating the enzyme immobilizates with a solution of the siloxane, for example a solution of the siloxane in an organic solvent, especially cyclohexane or toluene. Subsequently, the solvent can be removed, for example, by evaporation. The concentration of siloxane in such a solution is preferably 10 to 100% by mass, more preferably 30 to 100% by mass. However, preference is given to obtaining the inventive silicone coating by carrying out the hydrosilylation in the presence of the enzyme immobilizates.

The inventive enzyme preparations are preferably produced by the process according to the invention described below. This process for producing enzyme preparations is notable in that enzyme immobilizates are provided with a silicone coating obtained by hydrosilylating a self-crosslinking polysiloxane.

In the process according to the invention, the self-crosslinking polysiloxane used is preferably a compound of the general formula (I) as described above. Preference is given to using the polysiloxanes, which are used with preference in the inventive enzyme preparations.

Preference is given to performing the process according to the invention in such a way that the enzyme immobilizates are provided with a silicone coating by contacting the enzyme immobilizates, under hydrosilylation conditions, with a reaction mixture which comprises at least one polysiloxane of the general formula (I) and a catalyst which catalyses the hydrosilylation. In particular, the process can be performed in such a way that a hydrosilylation reaction is carried out in the presence of enzyme immobilizates which comprise enzymes or microorganisms comprising enzymes immobilized on an inert support. The silicone, which forms in the hydrosilylation, can provide the enzyme immobilizate with a silicone coating.

The hydrosilylation can be carried out in a manner known to those skilled in the art. Preference is given to performing the hydrosilylation using the above-mentioned parameters/feedstocks/catalysts.

A further embodiment of the process according to the invention differs from the aforementioned embodiment in that the enzyme immobilizates to be coated are immersed into the desired reaction mixture, then removed from the reaction mixture and dried. The removal can be effected, for example, using a screen which retains the enzyme immobilizate particles. The immersion time is preferably 1 to 10 minutes. The drying can be effected in a conventional drying cabinet. Preference is given to effecting the drying/hardening at a temperature of 20° C. to 80° C., preferably at 40° C. to 60° C., more preferably at approx. 50° C.

In a further embodiment of the process according to the invention, which is suitable especially for performance on the industrial scale, the hydrosilylation is carried out using a pelletizing pan unit (for example, from Erweka or Eirich). In this case, a defined amount of enzyme immobilizate particles is added to the so-called pan unit and stirred. Subsequently, either the mixture comprising at least one polysiloxane of the general formula (I), and also catalyst and optionally solvent, is added, or else, preferably, using a two-substance nozzle (for example from Schlick or others), in which the mixture or the components are applied under pressure (for example nitrogen or synthetic air) in the form of a fine mist of droplets, in order to ensure a very substantially homogeneous distribution on the particles. After a prolonged coating time, the particles are removed as described above and dried or hardened in a drying cabinet at a temperature of 20° C. to 80° C., preferably of 40° C. to 60° C., more preferably of 50° C., for several hours, and can then be stored at room temperature until further use.

In a further embodiment, the particles can be generated in a fluidized bed reactor (for example from Erweka), in which particles and the reaction mixture are applied in appropriate mixing ratios with strong dispersion.

The inventive enzyme preparations can be used, for example, as biocatalysts, especially as industrial biocatalysts.

The examples which follow are intended to illustrate the present invention in detail without restricting the scope of protection which is evident from the description and the claims.

### Examples

Novozym 435 (NZ435) is a commercial enzyme immobilizate from Novozymes A/S, Bagsvaerd, Denmark, of a lipase B from C antarctica immobilized on a polymethacrylate by adsorption.

General Method for Determining the Synthesis Activity of Inventive and Noninventive Enzyme Immobilizates in PLU Units (Propyl Laurate Synthesis Activity in Solvent-Free System):

For comparative examination of enzyme preparations, 10 mg of catalytically active particles were added to 5 ml of equimolar substrate solution (lauric acid and 1-propanol) and incubated while shaking and/or stirring at 60° C. Samples (Vₛₐₘₚₗₑ: 50 μl) were taken every 5 min over 25 min and transferred into 950 μl of decane (internal standard: 4 mM dodecane). The PLUs were determined with reference to the initial product formation rates. Propyl laurate was detected by gas chromatography (retention time: 9.791 min) (Shimadzu 2010, BTX column from SGE; length 25 m, I.D. 0.22 μm; film: 0.25 μm; detector type: FID at 300° C.; injector temperature 275° C. and injection volume 1 μl, split ratio 35.0; carrier gas pressure (helium) 150 kPa; temperature programme: start temperature 60° C., hold for 1.5 min, temperature rise 20° C./min, end temperature 250° C., hold for 2.5 min).

Determination of the Protein Desorption Stability of Conventional Enzyme Immobilizates

For the purpose of determining the desorption stability with regard to the protein on the particle surface under harsh reaction conditions, fractions of 50 mg of NZ435 were shaken in 20 ml of MeCN/H₂O (1:1, v/v) solution at 45° C. for 30 min. As described in EP 2 011 865 A1, this leads to complete desorption in the case of adsorptively bound enzymes. The particles were recovered by means of a fluted filter, washed with 100 ml of H₂O, and dried at 50° C. for 12 h, in order to determine the synthesis activity in PLU according to the scheme described above. The results can be taken from Table 1. The loss of synthesis activity can be equated to the measure of protein desorption from the particle surface.

Production of an Inventive Enzyme Preparation.

1 g of NZ435 particles were admixed in a metal dish with a reaction mixture consisting of 1 g of at least one compound of the general formula I (these compounds are prepared by processes familiar to the person skilled in the art, as described, for example, in U.S. Pat. No. 7,196,153 B2, by equilibration), and Karstedt catalyst (Syloff 4000, product from Dow Corning, USA). The silicone component including the catalyst was dissolved in each case in 3 ml of cyclohexane before application and then added to the particles in the metal dish. The addition was immediately followed by vigorous dispersion by means of a vortexer (Ika, level 9) for 15-30 min until a majority of the cyclohexane had evaporated. Subsequently, the particles were dried at 50° C. in a drying cabinet for about 12 h.

Comparison of the Protein Desorption Stability of Uncoated NZ 435 with Inventive Enzyme Preparation

The desorption of the enzyme from the carrier surface can be characterized as described above by a leaching experiment with an MeCN/H₂O mixture. Table 1 first summarizes the synthesis activity of native NZ435 and some enzyme preparations coated in accordance with the invention. It is found that the enzyme preparations, compared to uncoated NZ435, retain between 78 and 86% of the synthesis activity after the silicone coating with the one-component system.

While untreated, native enzyme immobilizate no longer exhibits any measurable synthesis activity after incubation with MeCN/H₂O, the coated enzyme immobilizates after leaching achieve a residual activity of up to 92%.

TABLE 1
Synthesis activity of the enzyme preparations before and after the protein desorption

| | Synthesis act. | Act. yield | Synthesis act. | Residual activity |
|---|---|---|---|---|
| | [PLU/g NZ435] | [%] after | [PLU/g NZ435] | [%] after protein |
| Enzyme preparation | without desorption | coating | after desorption | desorption |
| Native NZ 435, without | 6400 | | 0* | 0* |
| silicone coating | | | | |
| NZ 435, coated with | 5300 | 83 | 4900 | 92 |
| 50%(m/m) | | | | |
| M^{V}₂D^{H}₄D₁₄₄** | | | | |
| NZ 435, coated with | 4000 | 63 | 3700 | 93 |
| 50%(m/m) | | | | |
| M^{V}₂D^{H}₄D₄₉₄** | | | | |
| NZ 435, coated with | 5500 | 86 | 4500 | 82 |
| 50%(m/m) | | | | |
| M₂D^{V}₄D^{H}₄D₁₉₀** | | | | |
| NZ 435, coated with | 5000 | 78 | 4100 | 82 |
| 50%(m/m) | | | | |
| M₂D^{V}₄D^{H}₄D₂₉₀** | | | | |

In the above-described activity test, no activity was quantifiable.
**R¹ = methyl, R² = vinyl.

While the present invention has been particularly shown and described with respect to preferred embodiments thereof, it will be understood by those skilled in the art that the foregoing and other changes in forms and details may be made without departing from the spirit and scope of the present invention. It is therefore intended that the present invention not be limited to the exact forms and details described and illustrated, but fall within the scope of the appended claims.

What is claimed is:

1. An enzyme preparation comprising an enzyme immobilizate with a silicone coating thereon, said coating is obtained by hydrosilylating a self-crosslinking polysiloxane.

2. The enzyme preparation according to claim 1, wherein the self-crosslinking polysiloxane is a compound of general formula (I)
MₐM^{H}_{b}M^{V}_{c}M^{R}_{d}DₑD^{H}_{f}D^{V}_{g}D^{R}ₕTᵢT^{H}ⱼT^{V}ₖT^{R}ₗQₘ (I)
where
M=[R¹₃SiO_{1/2}],
M^{H}=[HR¹₂SiO_{1/2}],
M^{V}=[R²R¹₂SiO_{1/2}],
M^{R}=[R³R¹₂SiO_{1/2}],
D=[R¹₂SiO_{2/2}],
D^{H}=[HR¹SiO_{2/2}],
D^{V}=[R²R¹SiO_{2/2}],
D^{R}=[R³R¹SiO_{2/2}],
T=[R¹SiO_{3/2}],
T^{H}=[HSiO_{3/2}],
T^{V}=[R²SiO_{3/2}],
T^{R}=[R³SiO_{3/2}], and
Q=[SiO_{4/2}]
where
R¹=independently identically or differently, selected from saturated or unsaturated, unbranched or branched alkyl groups having 1 to 30 carbon atoms, alkaryl radicals having 7 to 30 carbon atoms, and aryl radicals having 6 to 30 carbon atoms,
R²=identical or different radicals of general formula (II) 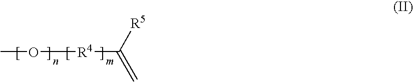
where
R⁴=identical or different, linear or branched alkanediyl radicals, optionally interrupted by one or more ether functions,
where
R⁵=R¹ or H,
where
n, m=0 or 1,
and
R³=identical or different polyether radicals,
where
a=0-30,
b=0-30,
c=0-30,
d=0-30,
e=0-800,
f=0-30,
g=0-30,
h=0-40,
i=0-10,
j=0-10,
k=0-10,
l=0-10, and
m=0-10,
with the proviso that
b+f+j>1,
and
c+g+h>1.

3. The enzyme preparation according to claim 2, wherein R³=identical or different polyether radicals of general formula (III)
—CH₂CH₂CH₂—[CH₂CH₂O]_{q}[CH₂CH(CH₃)O]ᵣ[CH₂CH(C₂H₅)O]ₛ[CH₂CH(Ph)O]ₜ—R⁶ (III)
where
R⁶ identical or different radicals from the group of:
H, R¹ and —C(O)—R¹
where q, r, s, t=0 to 50.

4. The enzyme preparation according to claim 1, wherein the enzyme immobilizate comprises enzymes selected from a class of hydrolases.

5. The enzyme preparation according to claim 1, wherein the enzyme immobilizate includes an inert support having a particle size distribution in which 90% of the particles have a particle size of 10 to 5000 μm.

6. The enzyme preparation according to claim 5, wherein the inert support comprises polyvinylstyrene, polymethacrylate or polyacrylate.

7. A process for producing enzyme preparations comprising providing a silicone coating to an enzyme immobilizate, said silicone coating is obtained by hydrosilylating a self-crosslinking polysiloxane.

8. The process according to claim 7, wherein the self-crosslinking polysiloxane is a compound of general formula (I)
MₐM^{H}_{b}M^{V}_{c}M^{R}_{d}DₑD^{H}_{f}D^{V}_{g}D^{R}ₕTᵢT^{H}ⱼT^{V}ₖT^{R}ₗQₘ (I)
where
M=[R¹₃SiO_{1/2}],
M^{H}=[HR¹₂SiO_{1/2}],
M^{V}=[R²R¹₂SiO_{1/2}],
M^{R}=[R³R¹₂SiO_{1/2}],
D=[R¹₂SiO_{2/2}],
D^{H}=[HR¹SiO_{2/2}],
D^{V}=[R²R¹SiO_{2/2}],
D^{R}=[R³R¹SiO_{2/2}],
T=[R¹SiO_{3/2}],
T^{H}=[HSiO_{3/2}],
T^{V}=[R²SiO_{3/2}],
T^{R}=[R³SiO_{3/2}], and
Q=[SiO_{4/2}]
where
R¹=independently identically or differently, selected from saturated or unsaturated, unbranched or branched alkyl groups having 1 to 30 carbon atoms, alkaryl radicals having 7 to 30 carbon atoms, and aryl radicals having 6 to 30 carbon atoms,
R²=identical or different radicals of general formula (II) 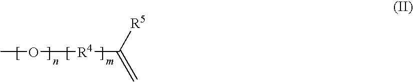
where
R⁴=identical or different, linear or branched alkanediyl radicals, optionally interrupted by one or more ether functions,
where
R⁵=R¹ or H,
where
n, m=0 or 1,
and
R³=identical or different polyether radicals,
where
a=0-30,
b=0-30,
c=0-30,
d=0-30,
e=0-800,
f=0-30,
g=0-30,
h=0-40,
i=0-10,
j=0-10,
k=0-10,
l=0-10, and
m=0-10,
with the proviso that
b+f+j>1,
and
c+g+h>1.

9. The process according to claim 8, wherein R³=identical or different polyether radicals of general formula (III)
—CH₂CH₂CH₂—[CH₂CH₂O]_{q}[CH₂CH(CH₃)O]ᵣ[CH₂CH(C₂H₅)O]ₛ[CH₂CH(Ph)O]ₜ—R⁶ (III)
where
R⁶=identical or different radicals from the group of:
H, R¹ and —C(O)—R¹
where q, r, s, t=0 to 50.

10. The process according to claim 7, wherein the enzyme immobilizate comprises enzymes selected from a class of hydrolases.

11. The process according to claim 7, wherein the enzyme immobilizate includes an inert support having a particle size distribution in which 90% of the particles have a particle size of 10 to 5000 μm.

12. The process according to claim 11, wherein the inert support comprises polyvinylstyrene, polymethacrylate or polyacrylate.

13. An industrial biocatalyst comprising the enzyme preparation according to claim 1.
